(19) United States
(12) Patent Application Publication
(10) Pub. No.: US 2005/0221464 A1
(43) Pub. Date: Oct. 6, 2005

(54) Method for culturing hcv virus in vitro

(75) Inventors: Patrice Andre, Lyon (FR); Florence Komurian-Pradel, Poleymieux (FR); Vincent Lotteau, Vourles (FR); Glaucia Paranhos-Baccala, Lyon (FR)

Correspondence Address: OLIFF & BERRIDGE, PLC, P.O. BOX 19928, ALEXANDRIA, VA, 22320, US

(73) Assignee: BIOMERIEUX, MARCY L'ETOILE (FR)

(21) Appl. No.: 10/515,381

(22) PCT Filed: Jun. 16, 2003
(86) PCT No.: PCT/FR03/01820

§ 371 Date: Nov. 23, 2004

(30) Foreign Application Priority Data
Jun. 18, 2002 (FR) 02/07600

(51) Int. Cl. C12N 7/02; C12N 5/08; C12N 15/86

(52) U.S. Cl. 435/239; 435/456; 435/366

(57) ABSTRACT

The invention concerns a method for HVC virus culture in vitro comprising steps which consist in: contacting particles containing hepatitis C virus RNA with cells capable of synthesizing and secreting lipoproteins, in a suitable culture environment promoting synthesis and secretion of lipoproteins, and in collecting the virus thus obtained. The invention has diagnostic and therapeutic applications.

The present invention relates to a novel method for culturing the hepatitis C virus in vitro.

Hepatitis C is the main cause of hepatitis acquired by transfusion. Hepatitis C can also be transmitted via other percutaneous routes, for example by intravenous injection of drugs. The risk of health professionals being contaminated is not, moreover negligible.

Hepatitis C differs from the other forms of virus-related liver diseases, such as hepatitis A, B or D. Infections with the hepatitis C virus (HCV) are often chronic, resulting in liver diseases such as hepatitis, cirrhosis and carcinoma in a large number of cases.

Although the risk of transmission of the virus by transfusion has decreased due to the selection of blood donors, the frequency of hepatitis C cases remains high. Currently, approximately 170 million people throughout the world are chronically infected with HCV. The high risk populations are found mainly among individuals who have undergone a blood transfusion and intravenous drug users, but asymptomatic blood donors exist who do not belong to these high risk groups and in whom circulating anti-HCV antibodies have been found. For the latter, the route of infection has still not been identified.

HCV was the first hepatotropic virus isolated by means of molecular biology techniques. The sequences of the virus genome were cloned without the viral particles having been visualized.

Currently, the only natural viral particles characterized in the blood of infected patients are nonenveloped capsids, even though it is known that other forms of particles containing the viral RNA exist.

For convenience, the term “virus” will be used in the present application to refer to any particle containing RNA of the HCV virus. The two terms will also be used without distinction.

HCV is a positive single-stranded RNA virus of approximately 9.5 kb which replicates by means of a copy of complementary RNA and the product of translation of which is a single polyprotein of approximately 3000 amino acids. The 5′ end of the HCV genome corresponds to an untranslated region adjacent to the genes which encode the structural proteins, the core protein of the nucleocapsid and the two envelope glycoproteins, E1 and E2. The 5′ untranslated region and the core gene are relatively well conserved in the various genotypes, but the E2 envelope proteins are encoded by a hypervariable region that is different from one isolate to another. The 3′ end of the HCV genome contains the genes which encode the nonstructural (NS) proteins and a well conserved 3′ noncoding region.

Because of its genomic organization and its presumed mode of replication, HCV has been classified in a new genus of the Flaviviridae family, the hepaciviruses.

The term “HCV virus” refers to any virus species, among which are the strains pathogenic for humans, the attenuated strains and the defective strains derived from said strains. Indeed, it is known that RNA viruses exhibit a high spontaneous mutation rate. Multiple strains can therefore exist, which may be more or less virulent. It is within the scope of those skilled in the art to identify such strains, for example by nucleic acid and/or peptide sequence homology with respect to a reference strain and/or by identifying a strain or an isolate with respect to morphological and/or immunological criteria.

Many techniques have been developed for diagnosing an HCV inspection. For example, diagnostic immunoassays have been carried out for detecting antibodies directed against HCV proteins in the sera of patients. The synthesis of cDNA by reverse transcription of the viral RNA and amplification by PCR have also been used to detect the HCV genome, such as indirect measurement of a potentially infectious virus in the sera of chronically infected humans or those of experimentally infected chimpanzees. Moreover, on the basis of gene cloning, hybridization techniques with a DNA probe have also been developed.

However, it is recognized that the existing diagnostic techniques lack sensitivity and/or specificity and/or suffer from difficulties of implementation. By way of example, with the probe hybridization method, it is impossible to distinguish between a virus with low infectious capacity and a virus with high infectious capacity. It is therefore necessary, to inoculate a chimpanzee with the virus which must be tested, and to test the resulting infection on the animal, but this is difficult to carry out.

It is therefore of foremost importance, from a public health point of view, to be able to develop specific, sensitive and practical methods for identifying and screening for HCV carriers. One of the solutions could be to produce an effective system for culturing HCV in vitro, which would make it possible to obtain propagation of the virus, in particular for studying its replication mechanisms, for testing neutralizing antibodies or antiviral agents, and likewise for developing biological materials, diagnostic assays and vaccine preparations. Indeed, although the complete sequence of HCV has been available since 1989 (Q. L. Choo et al., Science 244, 359 (1989)), the understanding of the life cycle and of the mode of replication of HCV has been hindered by the lack of a suitable system of in vitro culture. Ito et al. (J. Gen. Virol. 77: 1043-1054 (1996)) have indeed confirmed the maintenance of the replication of HCV in primary cultures of human hepatocytes obtained from patients carrying HCV and for whom the disease was chronically established, and suggested a passage of infection, but problems relating to the propagation of the virus remain (impossibility of long-term culture) and the system developed is limited by the need of a supply of human liver and the laborious nature of the technique. Moreover, to date, there is no general consensus as to the tropism of HCV, and not all the cell receptors for the virus have yet been identified.

Particles containing viral RNA that are very heterogeneous in terms of density are found in the plasma of HCV-infected patients. This heterogeneity of density of the particles containing viral RNA is attributed to their association in variable proportion with lipoproteins (Thomsen et al., 1993, Med. Microbiol. Immunol. 182:639). In the description of the present patent application, the inventors have called these hybrid particles LVPs (lipo-viro-particles). The distribution of each of these forms along a density gradient varies from one patient to another. Existing analyses of the low-density particles show densities covering those of LDLs (low density lipoproteins) and of VLDLs (very low density lipoproteins).

The nature of the LVPs containing viral RNA is not at this time precisely known.

Patent application WO 01/09289 describes a method for culturing viruses, such as HCV, in vitro, from at least one fraction of LVPs obtained from serum or plasma of an infected patient, using cells which have an endocytosis pathway relayed by at least one lipoprotein receptor and modulated by an activating agent. This method endeavors especially to promote the entry of the fractions into the cells in culture. However, the replication of the virus remains limited and must therefore be optimized.

Patent application WO 02/10353 describes complexes consisting of LVPs associated with human immunoglobulins, having a density of less than or equal to 1.063 g/ml, and containing mainly HCV virus RNA, contrary to known data (Hijikata et al., 1993, J. Virol., 1953-1958). In fact, Hijikata et al. showed that a high infectivity was found in chimpanzees in the presence of particles having a density of less than 1.06 g/ml, such that there could not be human immunoglobulins, which are very dense, in this type of low-density particles. These complexes constitute a particular form of the HCV virus which is very infectious.

That patent application also describes a method for culturing the HCV virus in vitro, from the LVP/immunoglobulin complexes, using cells comprising at their surface at least one type of immunoglobulin Fc fragment receptor or one type of receptor having the ability to bind immunoglobulins. Here again, this method promotes the entry of the complexes into the cells in culture, but it does not allow an abundant multiplication of the virus.

The present inventors have now found a novel method for culturing the HCV virus in vitro, which makes it possible to solve the above drawback, namely it uses cells capable both of causing the entry of the particles containing the HCV-RNA and of improving the replication and the production.

In fact, the present inventors have demonstrated, surprisingly, that the particles of HCV RNA, called LVPs:
are unit particles that are spherical overall; they are not therefore agglomerates of virions bound to normal lipoproteins, as had been suggested in the prior art,
contain apolipoproteins B and E and triglycerides: they therefore have a lipoprotein structure, and in particular a structure of the VLDL or chylomicron type, and
contain the capsid and the RNA of the virus: they therefore differ from the normal lipoproteins that are found in humans and constitute an unconventional form of the virus.

Since the LVPs are hybrid particles, i.e. lipoproteins containing viral components, the replication can be unexpectedly improved using cells capable of synthesizing the lipoproteins.

Thus, a subject of the present invention is a method for culturing the HCV virus in vitro, comprising the steps consisting in bringing particles containing the RNA of the hepatitis C virus into contact with cells having the ability to synthesize and to secrete lipoproteins, in a suitable culture medium that promotes the synthesis and the secretion of the lipoproteins, and in harvesting the virus thus obtained.

The expression “particles containing the RNA of HCV” is intended to mean viral particles such as, in particular, in the form of an LVP/immunoglobulin complex or present in the serum or the plasma of patients detected to be HCV-positive and containing such particles, and any inoculant containing the viral RNA, whatever the mode of preparation of the viral RNA in said inoculant.

The LVP/immunoglobulin complexes and the method for purifying them from a sample of plasma or of serum of a patient infected with HCV have already been described in patent application WO 02/10353.

However, that document neither described nor suggested the particular nature of these LVPs of a lipoprotein type, i.e. whether they possess triglycerides, apolipoprotein B and, optionally, apolipoproteins E.

Apolipoprotein B is a human protein which is associated with the endoplasmic reticulum membrane and which initiates the assembly of VLDLs, the introduction of triglyceride in the presence of microsomal triglyceride transfer protein, and also the assembly of VLDLs in the lumen of the endoplasmic reticulum. This apolipoprotein is provided in two forms, apo B 100 and apo B 48, and is synthesized in the liver and the intestine.

Apolipoprotein E can be synthesized by many cells and in particular by hepatocytes. Moreover, it can be acquired by the VLDLs in the bloodstream.

The cells used in the method of the invention are all cells that have the ability to synthesize and secrete lipoproteins.

These cells may be either primary cells or cell lines.

The term “cell line” refers to established lines that are immortalized spontaneously or by manipulation. In practice, to carry out a viral culture of interest, it is necessary to have permissive cells that are readily maintained in culture. The cell line is therefore preferably an established cell line or a cell line which results from immortalization by various methods. This may be carried out (i) by the establishment of a stable, established, continuous line by co-culturing permissive cells with tumorized permissive cells of the same nature, which are capable of multiplying indefinitely and of ensuring the propagation of the virus within the culture, the viral inoculation taking place within the culture, (ii) using primary cells infected with the virus which are then cocultured with permissive tumorized cells which ensure the propagation of the virus within the culture of the cell line thus established, (iii) by viral infection of a cell line, for example an immortalized B lymphocyte line, for example with the Epstein-Barr virus, or else (iv) by modification of the telomerase activity.

The expression “cells having the ability to synthesize and secrete lipoproteins used in the method of the invention” is intended to mean in particular cells which have this ability spontaneously and cells which have acquired this ability after induction in a culture medium which promotes the differentiation or redifferentiation of the cells, or after transfection of genes for synthesis of the apolipoprotein apo B and of the microsomal triglyceride transfer protein MTP.

Examples of cells which have the ability to synthesize and secrete lipoproteins that are suitable for the purposes of the invention comprise intestinal epithelial cells of the enterocyte type, brain cells and liver cells.

According to one embodiment, the cells used are cells which have acquired the ability to synthesize and secrete lipoproteins after induction in a medium that promotes the differentiation or redifferentiation of the cells.

Indeed, some cells, such as liver cells, have lost their ability to produce lipoproteins, or other cells, such as intestinal epithelial cells, have an ability which can be improved. To overcome this drawback, it is possible to induce differentiation or redifferentiation of these cells by bringing them into contact with a suitable medium that promotes this differentiation or redifferentiation.

According to a particular embodiment, the cells that are suitable for the purposes of the invention are intestinal epithelial cells, and in particular Caco-2 cells (Van Greevenbroeck, M. M. J., et al., 2000, Atherosclerosis, 25-31).

In order to promote their ability to synthesize and secrete lipoproteins, the intestinal epithelial cells, such as Caco-2, can be pre-cultured for 3 weeks with a DMEM medium supplemented with 10% of fetal calf serum, in dishes coated with collagen or under semipermeable membranes.

By way of examples of liver cells, mention may be made of hepatocytes (Moshage, H., et al., 1992, Journal of Hepatology, 15, 404413) and hepatocarcinomas such as Hep G2 cells (Gherardi, E., et al., 1992, Journal of Cell Science, 103, 531-539).

According to another particular embodiment, the cells used in the method of the invention are hepatocarcinoma cells.

However, in this case, their ability to synthesize and secrete lipoproteins can be promoted by bringing these cells into contact with a modified DMEM medium, i.e. containing at least one agent for inducing lipoprotein synthesis. These cells are preferably Hep G2 cells.

As an agent for inducing lipoprotein synthesis, mention may be made of various lipids, such as fatty acids, preferably C₁₈ or C₂₀ fatty acids, for example oleate (Luchoomun, J., et al., 1999, The Journal of Biological Chemistry, Vol 274(28), 19565-19572), and phospholipids such as lysophosphatidylcholine (Zhou, Z., 1998, Biochimica et Biophysica Acta, 1391, 13-24).

The use of oleate in the hepatocarcinoma redifferentiation medium constitutes a particular embodiment of the invention.

According to one embodiment of the invention, the modified DMEM medium consists, besides of DMEM (Gibco BRL) and an agent for inducing lipoprotein synthesis, of 1% HEPES (Gibco), of 1% glutamine (Gibco), of 0.25 mg/ml of gentamycin (Gibco), of 1.5% Ultroser-G (Gibco), of 5×10⁶ M forskolin (ICN), of 1.6×10⁻⁷ M PMA (4-α-phorbol 12-myristatel13-acetate) (Sigma), of 5.6 IU/ml of retinol acetate (Sigma), of 0.5×10⁻³ M sodium butyrate (Sigma), of 10⁻² M niacidamide (ICN), of 2×10⁻⁶ g/ml of polybrene. (Sigma), of 2.9×10⁻⁸ M sodium selenite (ICN) and of 1×10⁻⁹ M triiodo-L-thyronine sodium salt (ICN).

Another type of cells that can be used in the method of the invention consist of the cells obtained after transfection of genes for synthesis of the apolipoproteins apo B and, optionally, apo E and of the microsomal triglyceride transfer protein. By way of example of such cells, mention may be made of transfected insect cell lines as described by D. G. Gretch, et al., in The Journal of Biological Biochemistry, 1998, Vol 271(15), 8682-8691.

The particles containing the HCV RNA, when they are brought into contact with the cells that have the ability to synthesize and secrete lipoproteins, enter into these cells either via the lipoprotein receptors of said cells, of the LDL receptor type, as in the case of the LVPs for example, or by means of internalization by transfection according to techniques known to those skilled in the art, as in the case of viral RNA preparations, for example.

The culture medium used in the method of the invention is thus that it promotes the synthesis and the secretion of lipoproteins, preferably of VLDL or chylomicron type.

A suitable medium that is selected is a modified DMEM medium supplemented with agents that promote the metabolism of the cell in culture, and also with at least one agent for inducing lipoprotein synthesis.

As agents that promote the metabolism of the cell, mention may be made of dexamethasone and insulin.

A preferred modified DMEM medium is as defined above and supplemented with dexamethasone and insulin.

As agents for inducing lipoprotein synthesis, mention may be made of various lipids, such as fatty acids, preferably C₁₈ or C₂₀ fatty acids, for example oleate, phospholipids such as lysophosphatidylcholine, and also 22- or 25-OH-cholesterol.

The use of oleate as agent for inducing lipoprotein synthesis, optionally in combination with 22- or 25-OH-cholesterol, constitutes another particular embodiment of the invention.

The virus thus cultured by means of the method of the invention can be harvested by various methods such as centrifugation, for example on a density gradient, and immunoprecipitation using anti-apo B and/or anti-apo E antibodies.

The invention also relates to a diagnostic composition comprising at least the viral particles obtained according to the method of the invention or a component thereof as source of antigen.

Those skilled in the art will readily determine the amount of viral particles to be used according to the diagnostic technique used.

The invention also relates to a method for screening for and/or selecting at least one antiviral molecule, comprising the step of bringing said antiviral molecule into contact in the culture medium during the method of culture of the invention.

The selection of the antiviral molecules is carried out by means of techniques well known to those skilled in the art, such as the reduction in the number of intracellular viral RNAs and/or of infectious viral particles secreted into the supernatant in the presence of varying concentrations of the various inhibitors.

These inhibitors may be nucleotide or nucleoside analogs, or inhibitors of viral proteases or of other molecules, which interfere with the functions of the other viral proteins.

However, the invention also opens up other therapeutic perspective in that it makes it possible to develop a therapeutic composition capable of qualitatively and/or quantitatively influencing the propagation and the replication, in vivo, of HCV, which composition is characterized in that it comprises, inter alia, an agent capable of modulating, repressing or inhibiting lipoprotein synthesis, such as, for example, an inhibitor of the microsomal triglyceride transfer protein.

The expression “agent capable of modulating, repressing or inhibiting lipoprotein synthesis” is intended to mean any molecule making it possible, respectively, to control, decrease or suppress the propagation and the replication, in vivo, of HCV.

These agents can be selected by screening from a pool of molecules having recognized pharmacological activity on lipid metabolisms.

By way of example of an agent capable of inhibiting lipoprotein synthesis, mention may be made of the microsomal triglyceride transfer protein inhibitor.

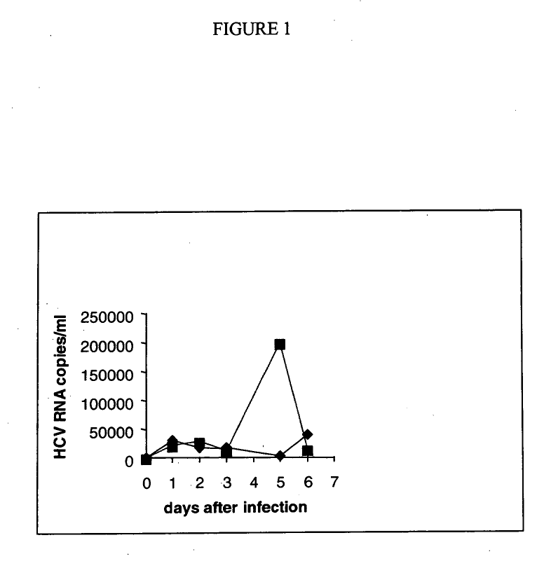

The invention will be understood more fully from the following examples given by way of nonlimiting illustration, and also from the attached FIGS. 1 to 3, in which:

FIG. 1 represents a graph showing the influence of the culture conditions (modified DMEM medium plus oleate compared with standard medium) for the Hep G2 cells on the secretion of viral HCV particles, which secretion is evaluated by quantifying the viral RNA in the culture supernatant as a function of time;

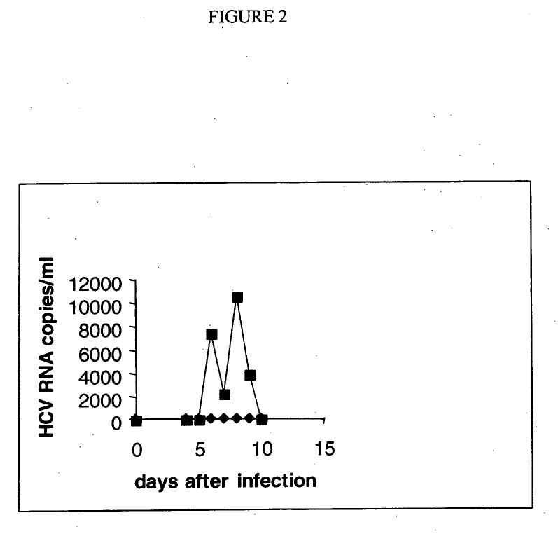

FIG. 2 represents a graph showing the influence of the addition of oleate to the modified DMEM culture medium on the secretion of viral particles, which secretion is evaluated by quantifying HCV RNA in the culture supernatant as a function of time, and

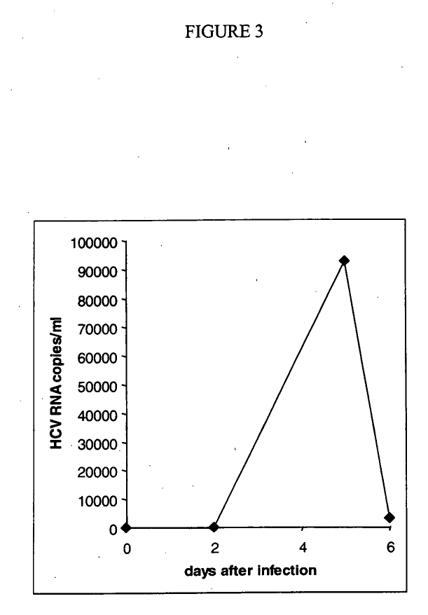

FIG. 3 represents a graph showing the HCV virus production by Caco-2 cells, after differentiation for 3 weeks of culture, said production being demonstrated by the number of viral RNAs in the culture supernatant as a function of time.

### EXAMPLE 1

### Characterization of LVPs

1.1 Demonstration of the Overall Spherical Form of the LVPs

The LVPs of sera from patients detected as being positive for the hepatitis C virus are purified as described in patent application WO 02/10353.

The low density (LDL) fraction, from which the purified LVPs had been removed, and also the purified LVPs, were diluted in PBS and were visualized using an electron microscope (JEOL device, Centre Commun d'Imagerie de Laennec, Lyon, France) after having floated drops of the sample on copper grids with a mesh size of 200, that were coated with a Formvar support film (Electron Microscopy Science, Pa.) for 3 min at ambient temperature, stained for 3 min by floating on a 4% (mass/vol) phototungstic acid medium, buffered at pH 7.2 with NaOH, and then dried.

The LDL fraction consisted of particles having a homogeneous spherical structure, with a mean diameter of 25 nm, in accordance with normal LDLs.

On the other hand, the purified LVPs were unusually large spherical structures, with a mean diameter of 100 nm.

1.2 Assaying of the LVP Constituents

a) Determination of the Lipid Concentration of the LVPs

The total cholesterol, phospholipid and triglyceride concentrations were determined using cholesterol RTU, phospholipids enzymatic PAP 150 and triglyceride enzymatic PAP 150 kits (bioMérieux, Marcy l'Etoile, France) according to the manufacturer's recommendations, and by establishing standard curves.

b) Determination of the apo B Concentration of the LVPs

The concentration of apolipoprotein apo B in the purified LVPs was determined by means of an ELISA assay. To do this, 96-well, flat-bottomed ELISA plates (Maxisorb; Nunc) were coated with 100 μl of monoclonal anti-human anti-apo B antibody (5 μg/ml; clone 1609; Biodesign, Saco, Me.) in PBS (phosphate buffer saline solution). The plates were left to stand overnight at 4° C., and then the reaction was blocked with 2% of BSA (bovine serum albumin).

The samples were first of all incubated for 30 min at ambient temperature in a mixture of PBS-0.2% BSA supplemented with 10 μg of human IgG/ml, and were then distributed onto the plates in a proportion of 100 μl/well.

After incubation for 2 h at 37° C. and washing with a PBS-0.05% Tween 20 medium, goat anti-human apo B antibodies conjugated to peroxidase (1.6 μg/ml; Biodesign), in a PBS-0.2% BSA mixture, were added in a proportion of 100 μl/well, and the plates were left to incubate for 90 min at 37° C.

The plates were washed and the o-phenylenediamine substrate (Sigma) was added in a proportion of 150 μl/well. The reaction was allowed to develop for 10 min and the plates were read at 490 nm.

c) Results

The results, in terms of triglyceride/cholesterol (TG/Chol) and triglyceride/apo B (TG/apo B) ratio, are given in the table below. By way of comparison, this table also contains these same ratios for the normal lipoproteins of the patient, i.e. the lipoproteins obtained after extraction of the LVPs.

TABLE

| | Normal lipoproteins | | Purified LVPs | |
|---|---|---|---|---|
| Fraction density | TG/Chol | TG/apo B | TG/Chol | TG/apo B |
| <1.006 | 3.4 ± 1.7 | 15 ± 1.3^{a} | 3.1 ± 1.6 | 160 ± 47^{a} |
| 1.025-1.055 | 0.45 ± 0.2^{b} | 0.8 ± 0.2^{a} | 2.8 ± 1.9^{b} | 26 ± 20^{a} |

^{a}p ≦ 0.04
^{b}p ≦ 0.01

The LVPs, which are found in the two fractions of different density, namely low and very low densities, contain more triglyceride per molecule of apo B than the normal lipoproteins of the same fractions.

These data confirm that:
the LVPs indeed contain lipids,
the lipid concentration of the LVPs is different from those of the normal lipoproteins; this therefore excludes any contamination, and
the LVPs contain apo B.

1.3 Presence of Apolipoproteins B and E

The presence of apo B and of apo E was demonstrated by preventing the entry of the LVPs into PLC cells after blocking the sites of binding to the apo B and apo E receptors of said cells as follows:

5×10⁵ PLC/PFR/5 human hepatoma cell line cells (ATCC CRL 8024) (Alexander cells) (human hepatoma) were cultured in 96-well plates (Maxisorb, Nunc) for 24 h with a DMEM culture medium (Gibco, BRL) supplemented with 10% of fetal calf serum (Biowhittaker, Emerainville, France), 2 mM of HEPES (Gibco/BRL), 1% of nonessential amino acids (Gibco/BRL) and 50 IU of penicillin/streptamycin (Gibco/BRL)/ml at 37° C.

Firstly, the apo B receptor-binding sites were blocked with monoclonal antibodies directed against the apo B receptor-binding site (4G3 and 5^{E}11; Ottawa Heart Institute Research Corporation, Ottawa, Ontario Canada) and, secondly, the apo E receptor-binding sites were blocked with monoclonal antibodies 1D7 (Ottawa Heart Institute Research Corporation). The PLC cells were washed three times with PBS and were incubated for 3 h with purified LVPs. The cells were washed and were then harvested in 350 μl of lysis buffer from the Rneasy kit (Qiagen), and the RNA was extracted as indicated above.

The blocking of the recognition of the LVPs by blocking the apo B and apo E recognition sites indicates that they contain apolipoproteins and demonstrates the lipoprotein structure of the LVPs.

1.4 Presence of the Capsid Consisting of the Core Protein and of the RNA of the Virus, in the LVPs

The presence of the capsid was demonstrated by delipidating the purified LVPs in the following way: the LVPs were incubated for 30 min, while gently stirring, in a solution of 85% of ether-15% of butanol. The presence of the capsid was visualized using an electron microscope (JEOL device, Centre Commun d'Imagerie de Laennec, Lyon, France) as indicated in point 1.1 above.

The presence of the HCV virus core protein was confirmed by Western blotting by bringing the LVPs that had been delipidated as described above into contact with anti-HCV core protein monoclonal antibodies (19D9D6; Jolivet-Reynaud, C. P., et al., 1998, J. Med. Virol., 56, 300-309) and 10 nm-gold labeled secondary antibodies, and visualizing using the grids indicated above, by immunodetection after negative staining by floating on 3% uranyl acetate.

Finally, the presence of the virus RNA was demonstrated by extraction of the purified and delipidated LVPs using a QIAamp kit (Qiagen S. A. Courtaboeuf, France).

### Example 2

### Influence of the Culture Conditions on the Production of HCV Virus by Hep G2 Cells

Hep G2 cells were first of all cultured either in a medium consisting of DMEM and of 10% fetal calf serum (standard medium), as in patent application WO 01/09289, or in a modified DMEM medium, i.e. containing DMEM supplemented with 1% HEPES, 1% glutamine, 0.25 mg/ml of gentamycin, 1.5% Ultroser-G, 5×10⁶ M forskolin, 1.6×10⁻⁷ MA, 5.6 IU/ml of retinol acetate, 0.5×10⁻³ M sodium butyrate, 10⁻² M niacidamide, 2×10⁻⁶ g/ml of polybrene, 2.9×10⁻⁸ M sodium selenite and 1×10⁻⁹ M triiodo-L-thyronine sodium salt.

Falcon 24-well culture plates were then seeded in a proportion of 150 000 cells per well with standard medium or modified DMEM medium, and left in culture for 24 h.

The medium was removed by suction and the cells were incubated in the presence of the virus, in a proportion of 500 000 HCV RNAs per well, for 6 h using DMEM supplemented with 0.2% of BSA.

The cells were then washed with PBS and were cultured either in standard medium, or in modified medium, as indicated above, but also supplemented with hexamethasone and insulin and a mixture of oleate and BSA in a proportion of 0.15 mM of oleate.

All the cultures were effected in a 5% CO₂ atmosphere and at 37° C.

A sample of supernatant was then taken from the wells (in triplicate) and the HCV RNA was quantified by RT-PCR according to the protocol described by Komurian-Pradel, F. in J. Virol. Methods, 2001, 95, 111-119.

The results, copies of RNA/ml of supernatant as a function of time, are given in FIG. 1, in which the diamonds represent the use of the standard medium and the squares the use of the medium for promoting the synthesis and secretion of lipoproteins.

This figure demonstrates that the replication of the virus and the secretion thereof are improved by using cells having the ability to synthesize and secrete lipoproteins and by placing them under favorable conditions.

### Example 3

### Importance of the Addition of Lipids to the Medium for Promoting the Synthesis and Secretion of Lipoproteins

Hep G2 cells were first of all cultured for 24 h in a modified medium as described in example 2 above, and were added to the wells of the Falcon 24-well culture plates in a proportion of 150 000 cells per well.

The cells were then incubated in the presence of the virus, in a proportion of 400 000 HCV RNAs per well, for 6 h in a DMEM medium supplemented with 0.2% of BSA, alone or with 0.1 μM of 25-OH-cholesterol.

The cells were then washed as indicated in example 2 above, and were cultured in modified medium.

Three days after the inoculation of the cells, an oleate-BSA mixture with 0.15 mM of oleate was added.

The supernatant was collected and the HCV RNA was quantified by RT-PCR.

The results, demonstrating the importance of the use of oleate, are given in FIG. 2 in which the diamonds represent the use of the modified medium alone and the squares the use of the modified medium supplemented with oleate and with 25-OH-cholesterol.

### Example 4

### Culturing of the Virus Using Caco-2 Cells

Caco-2 cells are differentiated on semipermeable membranes (Transwell, Costar) contained in 24-well plates, for 3 weeks in standard medium (DMEM supplemented with 10% of fetal calf serum).

The cells thus prepared were incubated with the viral particles in a proportion of 200 000 HCV RNAs/well, for 6 h.

The cells were washed and were cultured in DMEM medium supplemented with 10% of fetal calf serum and with oleate-taurocholate containing 0.15 mM of oleate.

The supernatant above the insert was taken, and the viral RNA was quantified by RT-PCR.

The results are given in FIG. 3, which demonstrates the culturing of the HCV virus by means of intestinal epithelial cells in a medium for promoting the synthesis and secretion of lipoproteins.

1. A method for culturing the hepatitis C virus in vitro, characterized in that it comprises the steps consisting in bringing particles containing the RNA of the hepatitis C virus into contact with cells having the ability to synthesize and to secrete lipoproteins, in a suitable culture medium that promotes the synthesis and the secretion of the lipoproteins, and in harvesting the virus thus obtained.

2. The method as claimed in claim 1, characterized in that the particles containing the RNA of the hepatitis C virus are LVP lipo-viro-particles.

3. The method as claimed in claim 1, characterized in that the cells that have the ability to synthesize and secrete lipoproteins are chosen from cells that spontaneously have this ability and cells that have acquired this ability after induction in a culture medium that promotes the differentiation or redifferentiation of the cells or after transfection of genes for synthesizing apolipoprotein B and the microsomal triglyceride transfer protein.

4. The method as claimed in claim 3, characterized in that the cells used are cells which have acquired the ability to synthesize and secrete lipoproteins after induction in a medium that promotes the differentiation or redifferentiation of the cells.

5. The method as claimed in claim 1, characterized in that said cells are intestinal epithelial cells.

6. The method as claimed in claim 5, characterized in that said cells are Caco-2 cells.

7. The method as claimed in claim 4, characterized in that the cells are pre-treated for 3 weeks with a DMEM medium supplemented with 10% of fetal calf serum.

8. The method as claimed in claim 1, characterized in that said cells are hepatocarcinoma cells.

9. The method as claimed in claim 8, characterized in that the hepatocarcinoma cells are Hep G2 cells

10. The method as claimed in claim 8, characterized in that said cells are brought into contact beforehand with a modified DMEM medium containing an agent for inducing lipoprotein synthesis.

11. The method as claimed in claim 10, characterized in that the agent for inducing lipoprotein synthesis is oleate.

12. The method as claimed in claim 1, characterized in that the medium that promotes the synthesis and secretion of lipoproteins is a medium derived from the modified DMEM medium, supplemented with agents for promoting the metabolism of the culture cell, and also with at least one agent for inducing lipoprotein synthesis.

13. The method as claimed in claim 12, characterized in that said medium comprises oleate as agent for inducing lipoprotein synthesis.

14. The method as claimed in claim 13, characterized in that said medium also contains 22- or 25-OH-cholesterol as another agent for inducing lipoprotein synthesis.

15. A method for screening for and/or selecting at least one antiviral molecule, characterized in that it comprises the step of bringing said antiviral molecule into contact in the culture medium during the method of culture as claimed in claim 1.

16. A therapeutic composition capable of qualitatively and/or quantitatively influencing the propagation and the replication, in vivo, of HCV, characterized in that it comprises, inter alia, an agent capable of modulating, repressing or inhibiting lipoprotein synthesis.
